(19) United States
(12) Patent Application Publication
(10) Pub. No.: US 2007/0238933 A1
(43) Pub. Date: Oct. 11, 2007

(54) Posterior approach retractor ring and attachments system

(75) Inventors: Red Alinsod, La Canada, CA (US); James M. Fowler JR., Houston, TX (US); Marc D. Gelnett, Houston, TX (US)

Correspondence Address: VINSON & ELKINS L.L.P., 1001 FANNIN STREET, 2300 FIRST CITY TOWER, HOUSTON, TX, 77002-6760, US

(73) Assignee: Lone Star Medical Products, Inc., Stafford, TX (US)

(21) Appl. No.: 11/401,772

(22) Filed: Apr. 11, 2006

(51) Int. Cl. A61B 1/32

(52) U.S. Cl. 600/231

(57) ABSTRACT

The present invention provides an open looped (open-ended) retractor frame for use in surgical procedures. The open-ended retractor frame of the present invention is particularly useful in infracoccygeal sacropexy or posterior intravaginal slingplasty procedures. The present invention furthermore provides a variety of clip-on attachments to the open-ended retractor frame that provide different functionalities.

### BACKGROUND OF THE INVENTION

1. Field of the Invention

The present invention relates to surgical retractors and stay or blade attachments thereto. The present invention specifically relates to an annular, semi-annular or open retractor frame adapted for receiving and securing an end portion of an elastic member of a surgical retractor stay or blade. More particularly, the present invention relates to a posterior approach retractor frame having an open end (i.e., the frame does not constitute a closed loop).

2. Description of Related Art

During the course of many surgical procedures, anatomical features or tissue surrounding an incision or wound is separated and retracted by means of retractors or stay sutures, which are generally held by trained assistants. Most retractors are one piece metallic implements that retract a wound in a non-yielding manner. Many retractors retract the wound in a manner such that manipulation and movement by the surgeon as well as movement caused by contracting muscles or tissues of the patient can result in bruising or tearing of the tissue. Once the tissue is separated and retracted, further stabilizing, retracting, or delivering of exposed tissues or organs, requires the placement of additional retractors or stay sutures. Nevertheless, the use of these sutures and attached devices can clutter the surgical field for the surgeon and can be difficult to maintain in their desired positions due to the variations in the skills of the operating staff, especially in cases where the surgery takes an extended period of time.

Several retractor systems have been developed that require less continual maintenance by the physician and staff. Many of these embody devices designed for a designated area of the body on which surgery is to be performed without causing undue visual or physical obstructions.

More recently, elastic surgical retractor systems have come into commercial use that include elastic stays, each having an elongated elastic member that is typically a hollow length of elastic tubing. The elastic tubing provides proximal and distal end portions. The distal end portion typically carries an elongated hook constructed of wire which is placed in the distal end of the bore of the hollow tubing. In many instances, a shrink wrap is placed over the hook and tubing to hold the proximal end of the wire hook firmly in position within the bore of the tubing at the distal end. The embedded portion of the wire hook member is usually recurved or folded. This folded proximal portion of the wire hook can expand the tubing slightly, forming a vertically extended portion that defines a handle.

Various patents have issued for elastic stay retractor systems. A surgical retractor array system is disclosed in U.S. Pat. No. 4,434,791, issued to W. Dale Darnell on Mar. 6, 1984. This surgical retractor system comprises an array of standardized, interchangeable, annular retractor frame sections of various shapes of which the end portions are configured to permit the interchangeable, hinged connection of the various shaped frames in forming generally annular retractor units adaptable to conform to fit the surface contours of various patients upon which a surgical operation is to be performed. This retractor frame is designed to accept yielding rubber or like elastic stays.

Other patents have issued that relate to elastic type retractor stays and related retractor frames and systems. For example, U.S. Pat. No. 4,274,398, issued to Frank B. Scott, Jr., on Jun. 23, 1981, which is hereby incorporated by reference, discloses a surgical retractor which includes an annular frame conformed to fit the surface contour of the portion of the body to be operated on. At least one stay includes an elastic member and a tissue holding hook. The frame has a plurality of notches spaced about its periphery. The elastic portion of the stay is in the form of a length of hollow elastic tubing adapted to be inserted into one of the notches of the frame and held in place by friction to retract the tissue. The hook is a single, curved wire member. It has a folded proximal end that fits the hollow bore of the elastic tube.

U.S. Pat. No. RE 32,021, issued to Frank B. Scott, Jr., on Nov. 5, 1985, which is hereby incorporated by reference, discloses a surgical retractor which includes a frame conformed to fit the surface contour of the portion of the body to be operated on and at least one stay which includes an elastic member and tissue holding means. The frame, furthermore, has a plurality of notches spaced about its periphery and the elastic member of the stay is adapted to be inserted into one of the notches and held in place by friction to retract the tissue.

In addition, U.S. Patent Application Pub No. US2005/0171405A1, published on Aug. 4, 2005, which is hereby incorporated by reference, provides a surgical retractor system having an annular frame and at least one support member attached to the annular frame in a notch based attachment system, wherein at least a portion of the support member extends over at least a portion of the inner area of the frame.

Over the past decade, a new surgical technique has been developed for the treatment of vaginal vault prolapse that is only partially compatible with the use of the aforementioned annular (closed-loop) retractor frames. In vaginal vault prolapse, the upper part of the vagina sags into the vaginal canal typically because of a weakness in the anatomical supporting structures of the pelvis and vagina.

In cases of vaginal vault prolapse requiring surgical intervention, posterior infracoccygeal sacropexy or posterior intravaginal slingplasty (“posterior IVS”) is often the treatment of choice. Posterior IVS is designed to be a minimally invasive surgical technique where a tape or mesh is fed through a small puncture incision in one buttock, along one side of the vagina, across the top of the vagina, down along the other side of the vagina, and then back out a similar puncture incision in the other buttock. An incision is also made in the posterior wall of the vagina to allow the surgeon access to the tape/mesh for proper placement.

The supporting tape or mesh is introduced through the buttock incision and up around the vagina and back out the opposing buttock incision via a tunneling instrument. The utilization of the buttock incisions in this procedure drastically hinders a surgeon's ability to use an annular closed-loop retractor frame because the closed-loop frame interferes or blocks the surgeon's access to the buttocks puncture incision site.

Therefore, a need exists in the art for a retractor frame that is designed to increase a surgeon's access to the incision sites for posterior IVS procedures and/or provide easy access for a posterior weighted speculum. In addition, retractor frames typically have limited physical features to enhance suture and/or catheter attachment. In fact, such frames are most commonly used with the aforementioned stays or blades connected to an elastic tether which is secured to the retractor frame in a friction based manner or via a clamping structure. Nevertheless, during many, if not most, surgical procedures, the ability to quickly and temporarily attach a suture, or multiple sutures, to the retractor frame would be beneficial to the surgeon. Furthermore, many procedures involve the use not only of sutures but various tubing, such as catheter tubing, in a manner in which it would be advantageous to the surgeon to be able to securely attach tubing to the retractor frame in order to maintain the position of the tubing during a procedure.

### SUMMARY OF THE INVENTION

The present invention provides an open-ended retractor frame system for use in performing surgery that includes an opened looped structure having an inner edge, an outer edge, a distal side, a proximal side, and at least one blade attachment point. Certain other embodiments of the open-ended retractor frame system of the present invention include an open-ended retractor frame having two ends, a base, an inner edge, an outer edge, a proximal surface and a distal surface wherein the outer edge further includes a flanged portion that is thinner than but attached to the base and includes at least one notch and at least one blade attachment point.

In certain embodiments, the open-ended retractor frame can have a shape selected from the group consisting of: an open circle, an open oval, an open ellipse, an open rectangle and an open square. In still other embodiments, the open-ended retractor frame includes an opening between the ends of the open-ended retractor frame ranges from 3 to 6 inches or an opening between the ends of the open-ended retractor frame that extends 60° to 120°. In some embodiments, the open-ended retractor frame has an inner diameter that ranges from 6 to 8 inches.

In certain embodiments, the at least one blade attachment point is an attachment channel which may be used to attach a blade to the open-ended retractor frame with a screw. In certain of these embodiments, the screw head is counter-sunk to be flush with the distal side of the open-ended retractor frame when the blade is attached. In still other embodiments, the open-ended retractor frame further includes at least one drape attachment point on the flanged portion.

In still other embodiments, the open-ended retractor frame further includes a clip-on attachment connected to the open-ended retractor frame selected from the group comprising: a suture stay clip-on, a securing post clip-on and a catheter stay clip-on. Certain embodiments of the clip-on attachments of the present invention include an inner clip arm, an outer clip arm, a securing arm and at least one notch mating structure.

### DESCRIPTION OF THE FIGURES

FIG. 1a shows one embodiment of an open-ended retractor frame.

FIG. 1b provides a cross-sectional view of one embodiment of an open-ended retractor frame.

FIG. 1c provides a cross-sectional view of one embodiment of an open-ended retractor frame.

FIG. 2 shows an open-ended retractor frame utilizing elastic stays.

FIG. 3 provides a top and side view of an open-ended retractor frame.

FIG. 4 shows an open-ended retractor frame having a stabilizing cross-member.

FIG. 5 shows the open-ended retractor frame and an attachable blade/speculum.

FIG. 6 depicts one embodiment of a suture stay clip-on attachment.

FIG. 7 depicts a suture stay clip-on attachment having a compressive insert.

FIG. 8 depicts a suture stay clip-on attachment having a suture coding feature.

FIG. 9 depicts another embodiment of a suture stay clip-on attachment.

FIG. 10a depicts a securing bar clip-on attachment.

FIG. 10b depicts two securing bar clip-on attachments in conjunction with a connecting coil.

FIG. 11 depicts a catheter clip-on attachment.

### DESCRIPTION OF THE INVENTION

The retractor frame of the present invention is particularly useful in posterior IVS procedures, as well as Transvaginal, Suprapubic, and Transobturator slings, in Anterior compartment Repairs, Posterior Compartment repairs, Vaginal hysterectomies, and cervical procedures (LEEP, Cryosurgery, conizations), uterine procedures (hysteroscopy, endometrial ablations). In certain uses of the retractor frame of the present invention, the retractor frame can be used in an inverted position (“upside down”) such as with the myriad of slings currently used in surgical procedures. Positioning the retractor frame with the open end on top provides greater access to the suprapubic region for suprapubic slings, transvaginal slings, transobturator slings.

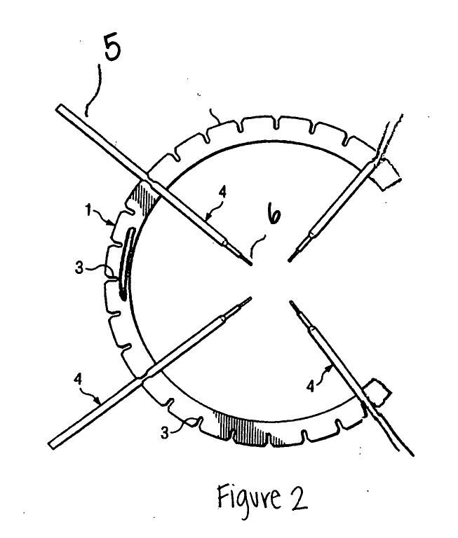

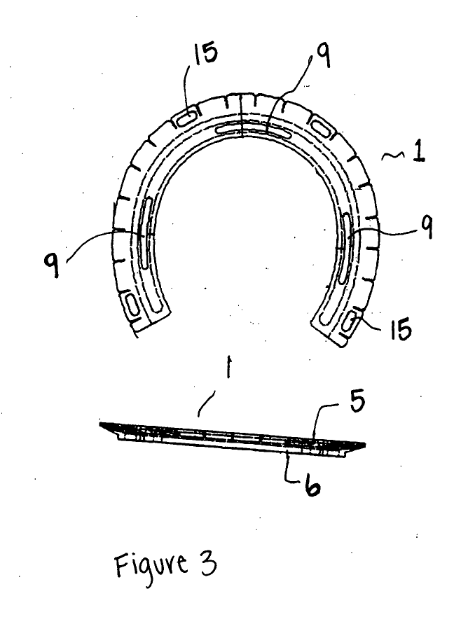

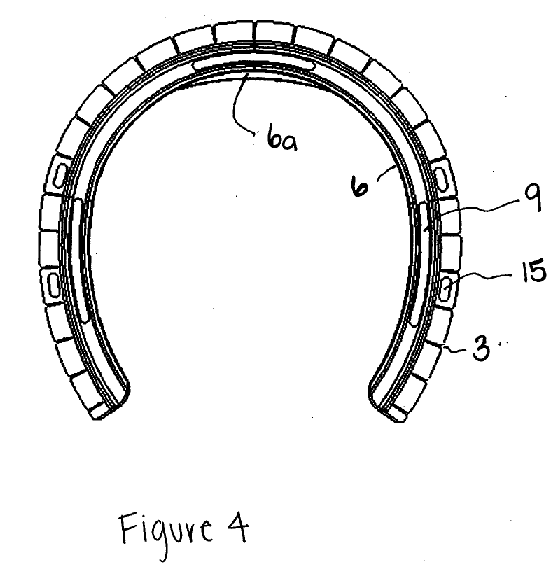

The present invention provides a retractor frame 1 having a generally open-ended (not a closed loop) 1a annular or semi-annular shape. In certain embodiments, the outer edge 2 of the retractor frame includes at least one, and preferably a plurality, of notches 3. The notches 3 are operable as connection points between the open-ended retractor frame 1 and a stay that includes an elastic tether. As shown in FIG. 2, in certain embodiments, the width and depth of at least one notch 3 is such that elastic portion 4 of a stay 5 may be inserted therein and held in place by friction without damaging the elastic portion 4. In such a configuration, the length of the elastic portion 4 of the stay 5 between the notch 3 and a tissue-holding device 6 located on the distal end of the stay will determine the amount of tension applied to the retracted tissue. Elastic tethered stays such as the one depicted in FIG. 2 are well known in the art and come in a wide variety of sizes and styles.

One of ordinary skill in the art will readily recognize that the open-ended retractor frame 1 of the present invention can be provided in any suitable size or configuration. In certain embodiments, the open-ended retractor frame 1 ranges from 5″ to 8″ across the inner diameter, with preferred embodiments having an inner diameter of 6″ to 7″. Certain embodiments can furthermore cover a range of 45° to 180° between the open ends of the retractor frame 1, with preferred embodiments ranging between 72° to 90°. Some embodiments of the open-ended frame 1 of the present invention can be wholly, or partially, circular, oval or elliptical in shape. Alternative embodiments, however, include retractor frames having squared or semi-squared corners thereby causing the open-ended retractor frame 1 to resemble an open rectangle or square.

The open-ended retractor frame 1 of the present invention can be formed of any suitable material. In certain embodiments, the frame is composed of a non-pliable material. In some embodiments, the open-ended retractor frame 1 is reusable and composed of a material that can be sterilized and re-sterilized, such as stainless steel, titanium, aluminum, or other suitable metals. In alternate embodiments, the open-ended retractor frame 1 is disposable and composed of a material such as a plastic material that is sufficiently strong to support the use of stays connected to it thereto without altering the shape of the open-ended retractor frame 1 (e.g. Noryl®, Lexan, etc.). Certain embodiments can be pre-sterilized and packaged for delivery to the surgeon.

Alternate embodiments of the present invention include an open-ended retractor frame 1 composed of suitable semi-pliable or malleable material(s). Suitable semi-pliable materials include materials that are capable of being molded into a particular shape while at the same time possessing enough resistance to shape changes to allow proper tension to be exerted on a retractor by an elastic stay or blade. Such materials include, but are not limited to, certain plastics and/or malleable metals (such as nitinol). In these embodiments, the shape of the retractor may be modified to correspond to the contour of the bodily region to be operated on.

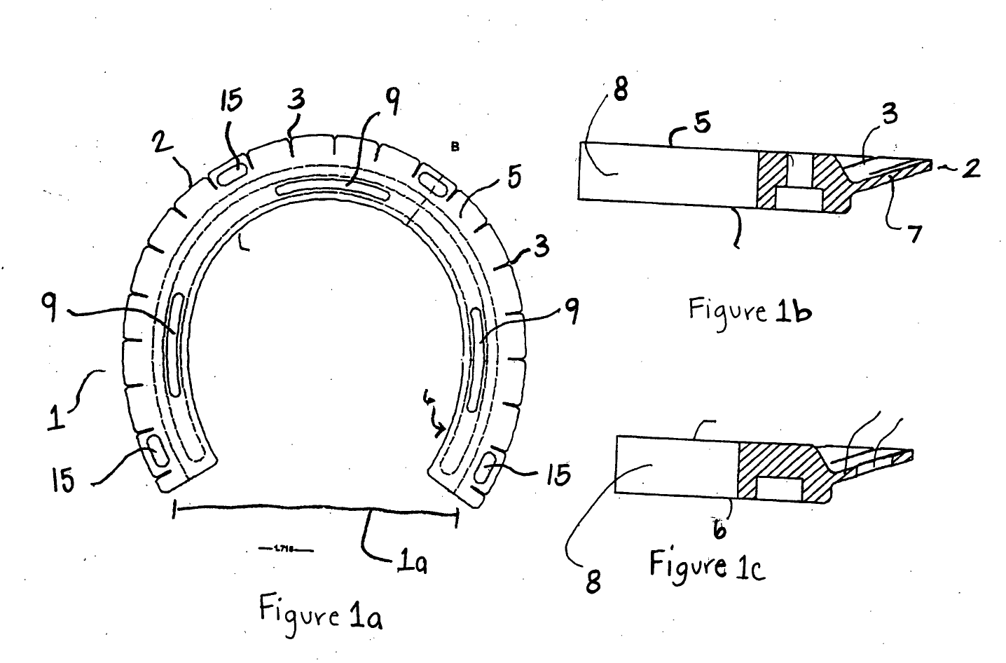

The open-ended retractor frame 1 further includes a proximal side 5 and a distal side 6 as shown in FIGS. 1b, 1c and 3. Certain embodiments of the open-ended retractor frame 1 include a bladed or flanged portion 7 that extends from the outwardly from the open-ended retractor frame 1 to form the outer edge 2. In some of these embodiments, the flanged portion 7 is thinner than the base 8 of the open-ended retractor frame 1 and at higher elevation than the distal side 6 of the base 8. In some embodiments, the outer edge 2 of the flanged portion 7 on the proximal side 5 is on the same plane as the proximal side of the base 8. The raised position of the flanged portion 7 as shown in FIGS. 1a and 1b make it and the notches 3 more accessible to the surgeon during a procedure (where the distal side 6 of the retractor rests on a patient's body) by providing an open access space between the patients body and the flanged portion. The open space allows the surgeon to readily attach and remove stays and sutures from the notches 3.

Certain embodiments of the open-ended retractor frame 1 include a stabilizing cross-member 6a, such as shown in FIG. 4. The stabilizing cross-member can be contiguous with the inner edge of the open-ended retractor frame 1 or can connect two or more discrete points or locations on the inner edge 6. The stabilizing cross-member 6a functions to increase the rigidity of the open-ended retractor frame 1.

In certain embodiments of the present invention, the open-ended retractor frame 1 also includes blade or speculum attachment points. One of ordinary skill in the art will readily recognize that such blade or speculum attachment points, as encompassed by the present invention, could be present in any number of configurations. For example, in certain embodiments, the attachment point is an attachment channel 9 designed to accommodate a screw 10 that in conjunction with a wing nut 11 can securely attach a blade 12 or speculum having a handle portion 13 to the open-ended retractor frame 1 (see FIG. 5). Some of these embodiments include at least one, or a plurality of, attachment points (such as attachment channels 9) positioned at different locations to increase the potential positioning of the blade 12 around the open-ended retractor frame 1. Other similar embodiments include an attachment channel 9 that extends from a position relatively close to one end of the open-ended retractor frame 1 around the length of the open-ended retractor frame 1 to a position near the other end of the frame. In other words, in these embodiments, the attachment channel 9 extends for the majority of the length of the open-ended retractor frame 1.

In some embodiments of the open-ended retractor frame 1 that include attachment channels 9, the open-ended retractor frame 1 further includes a groove 14 on the distal side 6 of the open-ended retractor frame 1 overlaying the channel(s) 9. This groove 14 is preferably designed as an off-set to allow the screw 10 head to be counter-sunk into the open-ended retractor frame 1 making it flush with, or even recessed within, the distal side 6 of the open-ended retractor frame 1.

Certain embodiments of the open-ended retractor frame 1 of the present invention include at least one, and preferably a plurality, of openings in the frame that function as drape attachment points 15 for towel clamps to attach the open-ended retractor frame 1 to a drape during use. In certain embodiments, the drape attachment points 15 are located on the flanged portion 7 of the open-ended retractor frame 1. One of ordinary skill in the art will readily recognize that the present invention encompasses a variety of physical features that would function adequately as drape attachment points on the open-ended retractor frame 1, including, but not limited to, loops, stems, bars, hooks, Velcro, adhesive patches or strips.

In certain configurations of the present invention that include an attachment point for a blade 12 or speculum, the outer edge 2 of the flanged portion 7 on the proximal side 5 is on the same plane as the proximal side 5 of the base 8 thereby increasing the stability of the blade 12/speculum attachment by providing at least two points of contact for the handle 13 of the blade 12 and the open-ended retractor frame 1 (one between the distal side of the handle 13 and the proximal side 5 of the base 8 of the open-ended retractor frame 1 and the other between the distal side of the handle 13 and the outer edge 2 of the flanged portion 7 on the proximal side 5).

One of ordinary skill in the art will readily recognize that the blade 12 or speculum used in conjunction with the open-ended retractor frame 1 can be designed in a wide variety of shapes and sizes, as well as from a variety of materials. In some embodiments, the blade 12 is reusable and composed of a material that can be sterilized and re-sterilized, such as stainless steel, titanium, aluminum, or other suitable metals. In alternate embodiments, the blade 12 is disposable and composed of a material such as a plastic material that is sufficiently strong to resist substantial deformation when used in connection with an open-ended retractor frame 1. Some of these embodiments can be pre-sterilized and packaged for delivery to the surgeon.

Certain embodiments of the blade 12 or speculum also have features designed to increase the stability (decrease the flexibility) of the blade 12 during use. For example, in some of these embodiments, the handle 13 portion includes ribs, braces, or similar structures molded into its design to increase its stiffness and thereby stability during attachment to the open-ended retractor frame 1.

Furthermore, in some embodiments, the open-ended retractor frame 1 of the present invention is designed to function with an assortment of clip-on attachments providing various functionalities. One such embodiment includes a clip that can be attached to the open-ended retractor frame 1 to act as an attachment point for a blade 12 or speculum. One of ordinary skill in the art will readily recognize a multitude of configurations that would allow a clip to act as an attachment point for a blade 12 or speculum. In some of these embodiments, the handle portion 13 of the blade 12 can be used for attachment to the clip-on. In certain of these embodiments, the clip-on includes a spring-loaded arm that can be opened such that the handle of the blade 12 fits under the arm and is securely held within the clip-on by the pressure of the spring. In some of these embodiments, the handle 13 of the blade 12 has a textured or serrated edge to increase the clips ability to securely hold the blade 12 in position.

Multiple mechanisms can be utilized to secure a clip-on attachment such as the blade clip described above to the open-ended retractor frame 1 of the present invention. FIG. 6 shows a clip-on attachment designed to function as a suture holder. Certain embodiments of the suture holder clip-on 16 include a base 16a that is roughly proportional to the width of the open-ended retractor frame 1 that it is intended to be used with, and include an inner clip arm 17 and an outer clip arm 18. The inner clip arm 17 extends down the inner edge 2a of the open-ended retractor frame 1 when attached, while the outer clip arm 18 extends down the outer edge 2. The outer clip arm 18, in some embodiments, also includes a securing arm 19. The securing arm 19 can be of any configuration that would enhance the stability of attachment to the open-ended retractor frame 1. Many such configurations include at least a portion of the securing arm 19 that extends under (on the distal side 5) of the outer edge 2 or flanged portion 7 but still allow the clip-on to be placed on and removed from the open-ended retractor frame 1. The embodiment depicted in FIG. 5 also includes a plurality of suture holding notches 20. When attached to the open-ended retractor frame 1, the surgeon can use the suture holder clip-on 16 to securely hold sutures extending from the wound or incision site to the open-ended retractor frame 1 by pulling the suture line into one of the suture holding notches 20. The suture holding notches 20 are typically slightly tapered having a narrowest point with a width less than that of the suture being used. Available suture widths are well known in the art and typically correspond to standardized sizes promulgated by United States Pharmacopeia.

FIG. 7 depicts yet another embodiment of a suture holding clip-on 16 attachment as provided by the present invention. The clip-on mechanism as depicted in FIG. 7 further includes a notch mating structure 21 in addition to an inner clip arm 17, an outer clip arm 18, and a securing arm 19. The notch mating structure 21 fits into at least one of the notches 3 on certain open-ended retractor frames 1. The interaction of the notch mating structure 21 with the notch 3 preferably increases the positional stability of the clip during use (at a minimum prevents the clip-on from sliding along the open-ended retractor frame 1). The combination of a notch mating structure 21 in addition to an inner clip arm 17, an outer clip arm 18, and a securing arm 19 can be utilized with any clip-on attachment regardless of the functionality it is intended to provide for the surgeon.

The clip-on attachment embodiment shown in FIG. 7 further includes a compressible insert 22 that includes at least one suture holding slot 23. In certain embodiments, the compressible insert 22 is composed of a soft plastic or silicon and suture holding slot(s) 23 have a width less than that of the width of the suture to be used with the clip-on attachment to provide an interference fit. In some of these embodiments, the compressible insert 22 is clear or slightly opaque and overlays a series of differentially colored or shaded bars (see FIG. 8) designed to be used as a coding system for each suture (i.e., the suture tying anatomical location is the one in the blue suture holding slot 23). One of ordinary skill in the art will readily recognize that multiple variations on the coding of particular suture holding slots 23 fall within the scope of the present disclosure. For example, the suture holding slots 23 can be numerically coded or the sections of the compressible insert 22 between the suture holding slots 23 can be shaded.

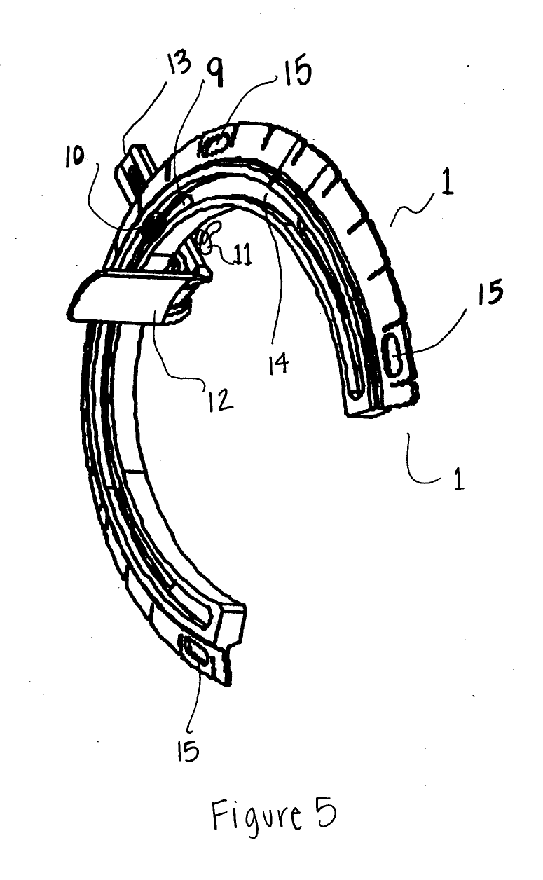

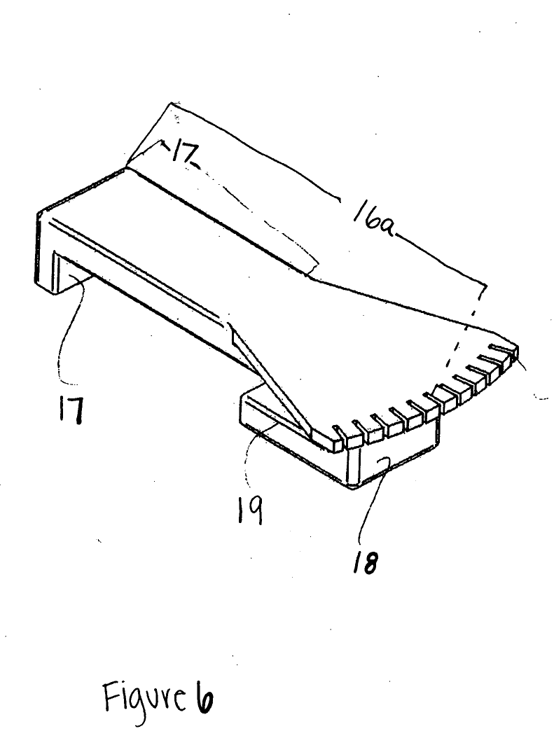

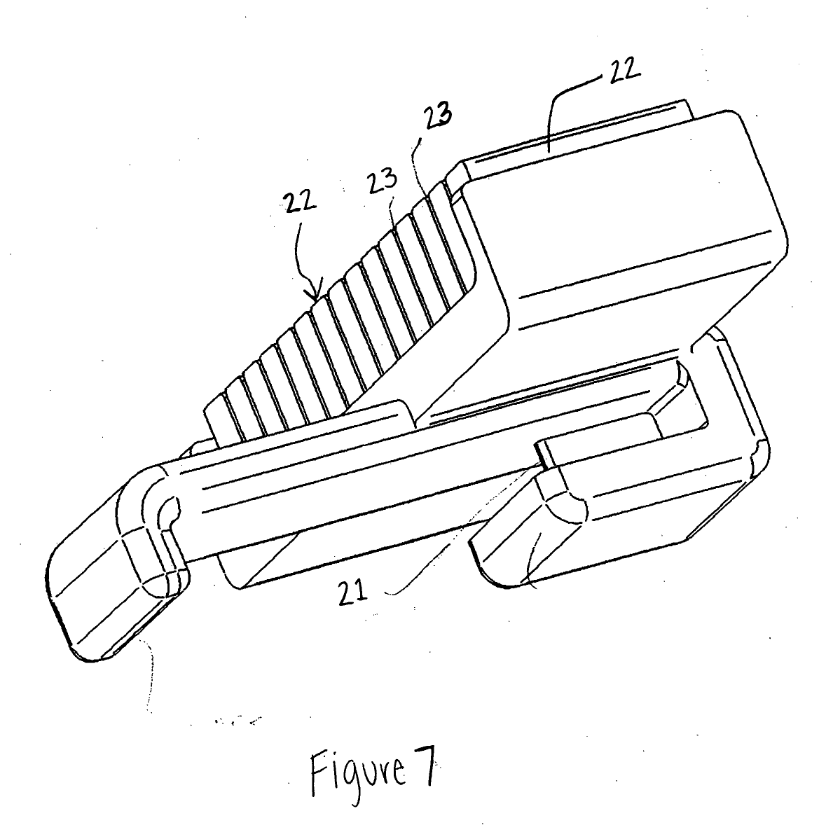

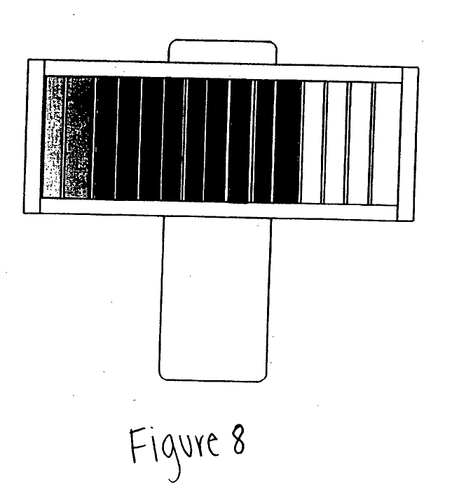

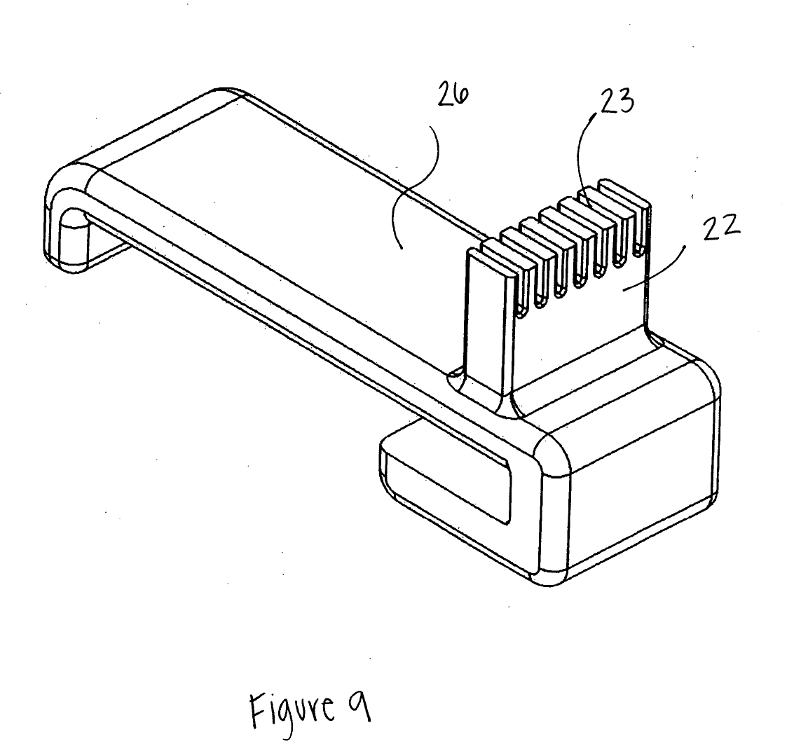

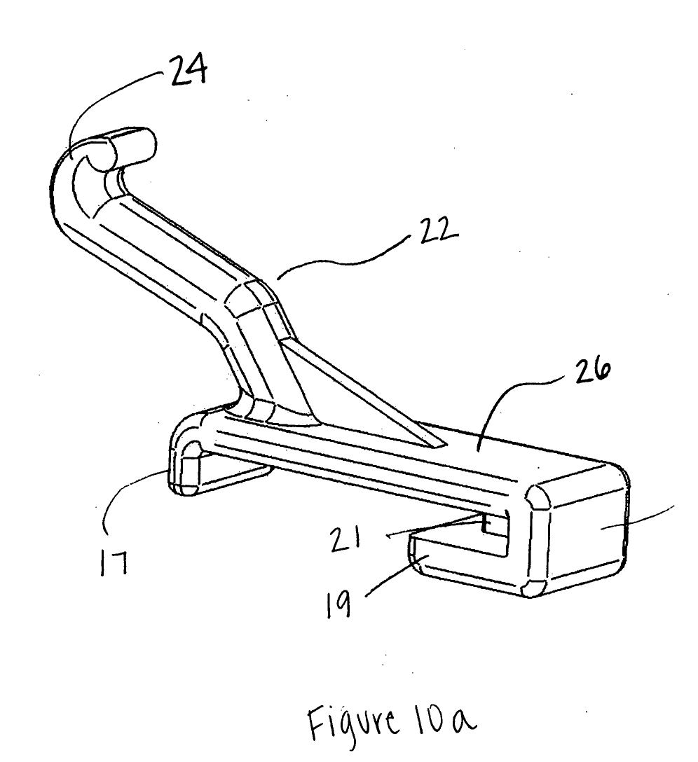

Multiple other configurations of clip-on attachments are also encompassed by the present invention. For example, FIG. 9 depicts another embodiment of a suture holding clip-on 16 attachment. This particular embodiment includes at least one, and preferably a plurality of, suture holding slots 23 are formed in a compressible insert 22 on the proximal side 26 of the attachment. Certain embodiments of this attachment also include an inner clip arm 17, an outer clip arm 18, and a securing arm 19 for connecting to the open-ended retractor frame 1. Some of these embodiments can also include notch mating structure 21.

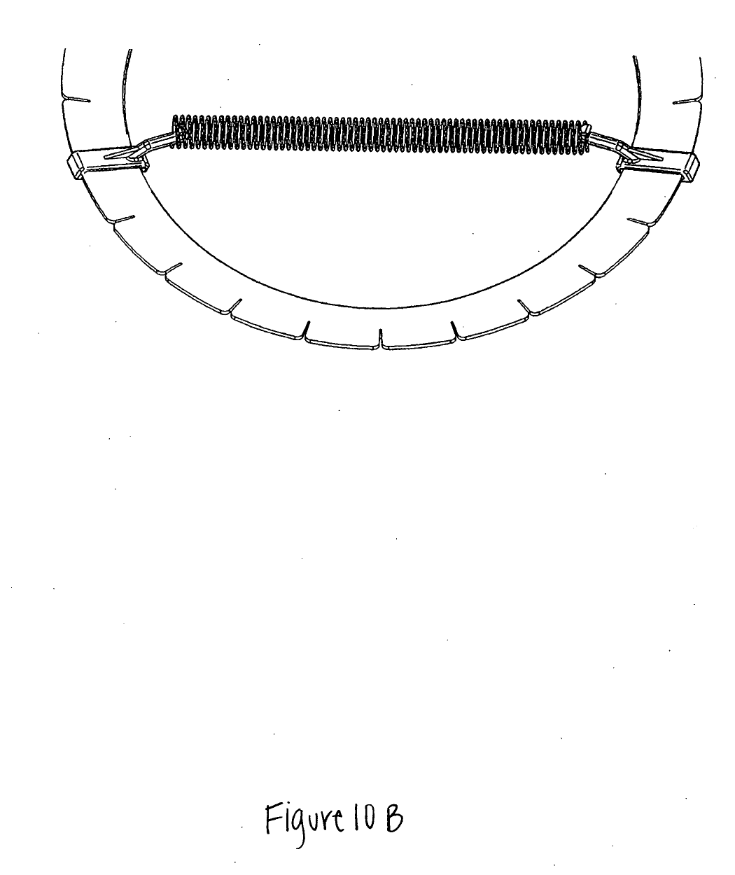

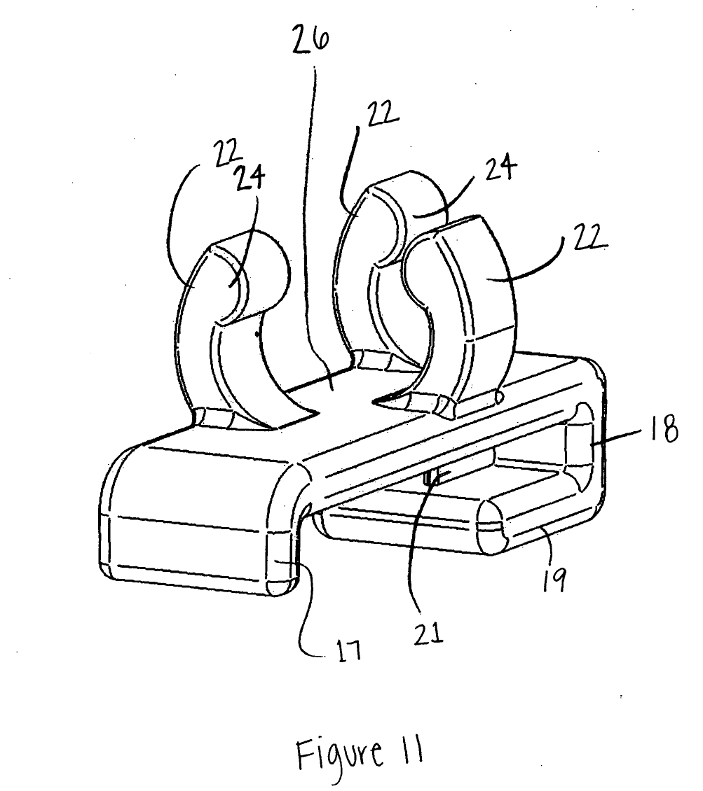

The present invention also includes a clip-on securing bar as depicted in FIGS. 10a and 10b. In some embodiments, the securing bar clip-on includes an inner clip arm 17, an outer clip arm 18, and a securing arm 19. Certain of these embodiments can also include a notch mating structure 21. The securing bar clip-on attachment further includes a securing post 22 that extends proximally toward the surgeon and away from the patient from the proximal surface 26 of the attachment. The securing post can be provided in a variety of configurations including, but not limited to, a simple post. Another configuration includes a securing post that not only extends proximally from the proximal surface 26 of the attachment but also extends inwardly out over the inner clip arm 17 and thus over the open inner circumference of the open-ended retractor frame 1 when attached thereto. In certain of these embodiments, the securing post 22 can also include a hook 24 or other similar structural feature.

The securing bar clip-on of the present invention can be used by the surgeon in a variety of fashions. Certain configurations can be used as suture stays wherein the surgeon simply wraps the suture line around the securing post 22. In still other functionalities, multiple securing bar clip-ons can be used in unison. FIG. 10b, for example, depicts two securing bar clip-ons being connected across the open inner circumference of a retractor frame with a coil 25. In certain embodiments, the coil 25 can be used as a suture stay by securing a suture line between two rings of the coil 25. The coil 25 can be composed of a variety of materials including metal or plastic. For example, an elastic stay or tubing with slits cut across it, or a silicon compressive insert, could be used to connect two securing bar clip-ons and provide the same functionality.

The present invention also includes a clip-on catheter or tube stay. In certain embodiments (such as the on depicted in FIG. 11), the catheter clip-on includes an inner clip arm 17, an outer clip arm 18, and a securing arm 19. Some of these embodiments can also include a notch mating structure 21. The catheter clip-on attachment further includes at least two securing posts 22 that extend proximally toward the surgeon (away from the patient) from the proximal surface 26 of the attachment such that a catheter tube can be held in between the securing posts 22. The securing posts 22 can be provided in a variety of configurations, including, but not limited to, a simple post. Another configuration includes at least two securing posts 22 that not only extend proximally from the proximal surface 26 of the attachment but also are curved to fit particular sizes of catheters. The securing posts 22 of some of these embodiments further includes a bulb 24 or other structural feature designed to hold the tube down in between the securing posts 22. The catheter stay clip-on attachment of the present invention can also be used as a stay for optic lighting tubes, endoscopic video feeds, and other tubular instruments or devices. Alternate embodiments of the catheter stay clip-on can have only two securing posts directly across from each other, a u-shaped clip (with or without bulbs to hold in a suture), a u-shaped clip on its side to accept the catheter from the side of the clip rather than the top.

One of ordinary skill in the art will readily recognize that the clip-on attachments of the present invention (discussed above) can be provided in a wide variety shapes and sizes, as well as be composed of a variety of materials. In some embodiments, an individual clip-on attachment can be reusable and composed of a material that can be sterilized and re-sterilized, such as stainless steel, titanium, aluminum, autoclavable Noryl, or other suitable metals. In alternate embodiments, an individual clip-on attachment is disposable and composed of a material such as a plastic material. Some of these embodiments can be pre-sterilized and packaged for delivery to the surgeon. Furthermore, the clip-on attachments of the present invention are designed to be used with any type of retractor frame and are not limited to use with the open-ended retractor frame disclosed herein.

From the foregoing, it can be seen that the retractor system of the present invention increases greatly the efficiency and effectiveness of the surgeon. Further modifications and alternative embodiments of the retractor system of this invention will be apparent to those skilled in the art in view of this description. Accordingly, this description is to be construed as illustrative only and is for the purpose of teaching those skilled in the art the manner of carrying out the invention. Various changes that may be made in the shape, size and arrangement of parts are encompassed by the correct disclosure.

What is claimed:

1. An open-ended retractor frame for use in performing surgery comprising:
opened looped structure wherein the open looped structure further comprises:
an inner edge;
an outer edge;
a distal side;
a proximal side; and
at least one blade attachment point.

2. The open-ended retractor frame of claim 1 wherein the outer edge includes at least one notch.

3. The open-ended retractor frame of claim 2 wherein the at least one blade attachment point is an attachment channel.

4. The open-ended retractor frame of claim 3 further comprising a blade attached to the open-ended retractor frame through the attachment channel with a screw.

5. The open-ended retractor frame of claim 4 wherein the attachment channel is overlaid with a groove that allows the screw to be flush with the distal side of the open-ended retractor frame when the blade is attached.

6. The open-ended retractor frame of claim 2 further comprising at least one drape attachment point.

7. The open-ended retractor frame of claim 2 further comprising a clip-on attachment connected to the open-ended retractor frame selected from the group comprising: a suture stay clip-on, a securing post clip-on and a catheter stay clip-on.

8. The open-ended retractor frame of claim 7 wherein the clip-on attachment further includes an inner clip arm, an outer clip arm, a securing arm and at least one notch mating structure.

9. The open-ended retractor frame of claim 2 wherein the open-ended retractor frame comprises a shape selected from the group consisting of: an open circle, an open oval, an open ellipse, an open rectangle, an open triable and an open square.

10. The open-ended retractor frame of claim 2 wherein the opening between the ends of the open-ended retractor frame ranges from 3 to 6 inches.

11. The open-ended retractor frame of claim 2 wherein the opening between the ends of the open-ended retractor frame extends 60° to 120°.

12. The open-ended retractor frame of claim 2 wherein the opening between the ends of the open-ended retractor frame extends 70° to 80°.

13. The open-ended retractor frame of claim 2 wherein the inner diameter of the open-ended refractor frame ranges from 6 to 8 inches.

14. The open-ended retractor frame of claim 2 further comprising a stabilizing cross-member connecting two points of the inner edge.

15. An open-ended retractor frame system comprising:
an open-ended retractor frame having two ends, a base, an inner edge, an outer edge, a proximal surface and a distal surface;
wherein the outer edge further comprises a flanged portion that is thinner than but attached to the base and includes:
at least one notch; and
at least one blade attachment point.

16. The open-ended retractor frame of claim 15, wherein the at least one blade attachment point is an attachment channel.

17. The open-ended retractor frame of claim 16 further comprising a blade attached to the open-ended retractor frame through the attachment channel with a screw.

18. The open-ended retractor frame of claim 17 wherein the attachment channel is overlaid with a groove that allows the screw to be flush with the distal side of the open-ended retractor frame when the blade is attached.

19. The open-ended retractor frame of claim 15 further comprising at least one drape attachment point on the flanged portion.

20. The open-ended retractor frame of claim 15 further comprising a clip-on attachment connected to the open-ended retractor frame selected from the group comprising: a suture stay clip-on, a securing post clip-on and a catheter stay clip-on.

21. The open-ended retractor frame of claim 20 wherein the clip-on attachment further includes an inner clip arm, an outer clip arm, a securing arm and at least one notch mating structure.

22. The open-ended retractor frame of claim 15 wherein the open-ended retractor frame comprises a shape selected from the group consisting of: an open circle, an open oval, an open ellipse, an open rectangle and an open square.

23. The open-ended retractor frame of claim 15 wherein the opening between the ends of the open-ended retractor frame ranges from 3 to 6 inches.

24. The open-ended retractor frame of claim 15 wherein the opening between the ends of the open-ended retractor frame extends 60° to 120°.

25. The open-ended retractor frame of claim 15 wherein the opening between the ends of the open-ended retractor frame extends 70° to 80°.

26. The open-ended retractor frame of claim 15 wherein the inner diameter of the open-ended refractor frame ranges from 6 to 8 inches.

27. The open-ended retractor frame of claim 15 wherein the proximal side of the base is on the same plane as the outer edge of the flanged portion.

28. The open-ended retractor frame of claim 27 further comprising a blade connected to the blade attachment point, wherein the blade also makes contact with the proximal side of the base and the outer edge of the flanged portion.

29. The open-ended retractor frame of claim 15 further comprising a stabilizing cross-member connecting two points of the inner edge.

30. A clip-on attachment for a annular or semi-annular retractor ring comprising:
a base having an inner end, an outer end, a top and a bottom;
an inner clip arm connected the inner end of the base;
an outer clip arm connected to the outer end of the base;
a securing arm connected to the outer clip are and extending back underneath the bottom of the base; and
a notch mating structure connected to the outer clip arm.

31. The clip-on attachment of claim 30 further comprising a compressible insert attached to the top of the base having at least one suture holding slot.

32. A clip-on attachment for a annular or semi-annular retractor ring comprising:
a base having an inner end, an outer end, a top and a bottom;
an inner clip arm connected the inner end of the base;
an outer clip arm connected to the outer end of the base;
a securing arm connected to the outer clip arm and extending back underneath the bottom of the base; and
a notch mating structure connected to the bottom of the base near the outer end.

33. The clip-on attachment of claim 32 further comprising a compressible insert attached to the top of the base having at least one suture holding slot.
